(19) United States
(12) Patent Application Publication
(10) Pub. No.: US 2011/0318815 A1
(43) Pub. Date: Dec. 29, 2011

(54) Method and System for Growing Microalgae in an Expanding Plug Flow Reactor

(76) Inventors: David A. Hazlebeck, El Cajon, CA (US); Xiaoxi Wu, Encinitas, CA (US)

(21) Appl. No.: 12/821,943

(22) Filed: Jun. 23, 2010

(51) Int. Cl. C12N 1/12; C12M 1/00

(52) U.S. Cl. 435/257.1; 435/289.1

(57) ABSTRACT

A method and system are provided for supporting the growth of algae cells. In the method, an inoculum of algae cells are grown in a closed bioreactor. Thereafter, the inoculum of algae cells is passed into an open system. Specifically, the inoculum is passed into an expanding plug flow reactor (EPFR) having an increasing width from its first to its second end. Further, medium is introduced into the EPFR to maintain a selected shallow depth. Importantly, the medium provides sufficient nutrients to support logarithmic growth of the algae cells to maintain a high concentration of algae cells, i.e., at least 0.5 grams per liter of medium, in the EPFR. After the desired level of growth is reached, the algae cells are transferred to a standard plug flow reactor wherein oil production is activated in the algae cells.

### FIELD OF THE INVENTION

The present invention pertains generally to methods for growing algae. More particularly, the present invention pertains to the use of an expanding plug flow reactor to reduce the requirement of using expensive closed system bioreactors for growing algae. The present invention is particularly, but not exclusively, useful as a method for growing algae in an open system comprising an expanding plug flow reactor fed with a medium to maintain a high concentration of algae cells.

### BACKGROUND OF THE INVENTION

As worldwide petroleum deposits decrease, there is rising concern over shortages and the costs that are associated with the production of hydrocarbon products. As a result, alternatives to products that are currently processed from petroleum are being investigated. In this effort, biofuel such as biodiesel has been identified as a possible alternative to petroleum-based transportation fuels. In general, a biodiesel is a fuel comprised of mono-alkyl esters of long chain fatty acids derived from plant oils or animal fats. In industrial practice, biodiesel is created when plant oils or animal fats are reacted with an alcohol, such as methanol.

For plant-derived biofuel, solar energy is first transformed into chemical energy through photosynthesis. The chemical energy is then refined into a usable fuel. Currently, the process involved in creating biofuel from plant oils is expensive relative to the process of extracting and refining petroleum. It is possible, however, that the cost of processing a plant-derived biofuel could be reduced by maximizing the rate of growth of the plant source. Because algae is known to be one of the most efficient plants for converting solar energy into cell growth, it is of particular interest as a biofuel source. Importantly, the use of algae as a biofuel source presents no exceptional problems, i.e., biofuel can be processed from oil in algae as easily as from oils in land-based plants.

While algae can efficiently transform solar energy into chemical energy via a high rate of cell growth, it has been difficult to create environments in which algae cell growth rates are optimized. Currently, the production of biofuel from algae is limited by a failure to maximize algae cell growth. Specifically, the conditions necessary to facilitate a fast growth rate for algae cells in large-scale operations have been found to be expensive to create. For instance, while providing high rates of algae cell growth, closed sterile environments such as inoculant tanks and controlled bioreactors are expensive to maintain and limited in scale. On the other hand, outdoor large-scale open systems, such as open runways, are plagued by contaminant organisms which fight the selected algae cells for nutrients and sunlight and reduce the rate of algae cell growth. Specifically, these contaminants include non-selected, i.e., “weed”, algae, viruses, bacteria, and grazers. Until now, it has been virtually impossible to prevent contaminant organisms from causing microbial instability and reducing selected algae cell growth rates in open systems. In fact, standard open systems typically provide only one to two days of microbial stability.

In light of the above, it is an object of the present invention to provide a method for minimizing the need for closed system inoculation of algae cells in a biofuel production system. Another object of the present invention is to maximize the cell growth rate of selected algae cells in an open system. Another object of the present invention is to provide an expanding plug flow reactor for supporting logarithmic growth of algae cells. Another object of the present invention is to selectively pump medium into the expanding plug flow reactor to maintain a high concentration of algae and a selected shallow depth of medium. Still another object of the present invention is to provide a method and system for growing selected algae cells in an open system in which contaminants cannot compete with the selected algae cells. Yet another object of the present invention is to provide a system and method for growing selected algae cells that is simple to implement, easy to use, and comparatively cost effective.

### SUMMARY OF THE INVENTION

In accordance with the present invention, a system is provided for growing selected algae cells in a medium and for preventing the growth of contaminants in the medium. In this endeavor, the system relies on the initial use of a closed reactor to grow an inoculum of microalgae. Importantly, the closed reactor is five times smaller than those used in known algae production systems. Specifically, the closed reactor comprises 0.4% of the present system while closed reactors typically comprise about 2% of known systems. For purposes of the present invention, the closed reactor is a continuous flow reactor such as a photobioreactor. Further, the closed reactor is designed to grow the inoculum of microalgae to a full concentration.

After the closed reactor grows microalgae to full concentration, the inoculum of microalgae is passed in an effluence to an open system. Specifically, the open system comprises an expanding plug flow reactor and a standard plug flow reactor. For the present invention, the expanding plug flow reactor continuously receives the effluence containing the inoculum of algae cells from the closed reactor. Further, the expanding plug flow reactor includes a conduit for continuously moving the effluence downstream under the influence of gravity with little back mixing. Preferably, the expanding plug flow reactor is an open raceway.

Structurally, the expanding plug flow reactor increases in width from its first end to its second end. Also, the expanding plug flow reactor is provided with a plurality of pumps along its length for introducing a growth medium to the conduit. Initially, the pumps dilute the effluence until the algae reaches a high concentration. For purposes of the present invention, “high concentration” is defined as at least about 0.5 grams per liter of fluid. Thereafter, as fluid evaporates and the algae cells grow, the pumps add growth medium to maintain the high concentration of algae. Further, the growth medium includes the nutrients necessary to support the desired growth of the algae cells.

Importantly, the pumps are controlled in response to the growth rate of the algae cells. For instance, the algae growth rate may decrease due to a reduction in the amount of sunlight received and lower air temperatures. As a result, in order to ensure a high concentration of algae as the expanding plug flow reactor widens, the pumps will provide less medium. Therefore, the depth of the medium will decrease slightly, and the flow rate of the algae cells will decrease due to the viscosity of the algae cells. With the reduced flow rate, the algae cells are provided with enough time to grow sufficiently to remain at a high concentration as the expanding plug flow reactor widens. Because the selected algae is maintained at a high concentration, the nutrients provided in the growth medium are rapidly consumed by the selected algae. As a result, the time available for growth of contaminants is limited.

When the selected algae cells reach the end of the expanding plug flow reactor, they have reached the desired level of growth. Thereafter, the algae cells are transferred to a standard plug flow reactor. Typically, the standard plug flow reactor will have the same width as the downstream end of the expanding plug flow reactor. Further, a trigger medium may be fed into the standard plug flow reactor to activate production of oil in the algae cells. Alternatively, no medium may be fed into the standard plug flow reactor. This alternative method is effective to trigger oil production because algae cells will convert stored energy to oil when being starved of certain, or all, nutrients. Further, as the medium evaporates in the standard plug flow reactor, the depth of the medium will be reduced until the algae naturally flocculates. In this manner, the standard plug flow reactor may be designed to self-flocculate when optimal oil production has been achieved.

For an alternate embodiment of the present invention, a system for growing algae cells includes a plurality of open ponds. In combination, open ponds in this plurality are connected for selective fluid communication with each other, and they are arranged in sequence from a first upstream pond to a last downstream pond. In a variation from the expanded plug flow reactor (EPFR) described above, this alternate embodiment of the invention establishes each downstream pond with an exponentially greater surface area relative to its adjacent upstream pond.

Structurally, the alternate embodiment of the present invention includes a first transfer conduit for transferring inoculum from an inoculum source into the first upstream pond. A culture is thereby created for algae growth in the first upstream pond. A subsequent transfer of the culture can then be made from the first upstream pond to successive downstream ponds for further algae growth. For the present invention, such transfers are periodically accomplished in a controlled manner, and algae is allowed to grow for a predetermined time in each of the successive ponds. Eventually, fully grown algae cells are transferred from the last downstream pond to an oil formation pond via a last transfer conduit.

Each open pond in the system, regardless of its relative size, will preferably have a fluid circulating device, such as a paddle wheel or circulation pump, that can be used to establish liquid flow in the pond. Preferably, each pond will also have a medium addition conduit for adding medium into the culture in the pond. Further, as envisioned for the present invention, the transfer of culture from an upstream pond to its adjacent downstream pond can be accomplished in either of two ways. For one, each pond may include a transfer pump for transferring the culture downstream from the pond to its adjacent downstream pond. For another, the ponds can be terraced so that a gravity flow can be established from an upstream pond to a downstream pond.

As implied above, a fixed multiplier is determined to establish a ratio of the surface areas for adjacent ponds. More specifically, the surface area of each pond relative to the surface area of an adjacent upstream or downstream pond will be established by this multiplier. In practice, the value of the multiplier may vary from system to system. Specifically, in each case the multiplier will be determined by the growth rate of the algae that is being used for cultivation in the particular system.

In an operation for the alternate embodiment of the present invention, a transfer sequence is periodically performed in accordance with a set procedure. Specifically, the transfer sequence is initiated by first transferring fully grown algae from the last downstream pond to an oil formation pond. Once this is done, and the last downstream pond has been emptied, culture from the adjacent upstream pond is then transferred into the now-empty, last downstream pond. As the culture is transferred, additional medium can also be transferred into the last downstream pond for further algae growth in the last downstream pond. The now-empty, immediately upstream pond can then receive culture transferred from its respective adjacent upstream pond. This process of transfer from an upstream pond to an emptied adjacent downstream pond continues until the first upstream pond has been emptied and subsequently refilled with inoculum from the source of inoculum. After an entire transfer sequence has been completed, the cultures in all of the open ponds are individually circulated to promote algae growth. Once algae growth in the respective ponds has been completed, the entire transfer sequence can then be repeated. Preferably, transfer sequences for the alternate embodiment of the present invention are accomplished during the nighttime.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic view of the system of the present invention, illustrating the flow of algae from the closed reactor, through the expanding plug flow reactor, and to the standard plug flow reactor in accordance with the present invention;

FIG. 2 is an overhead view, not to scale, of the expanding plug flow reactor shown in FIG. 1;

FIG. 3 is a longitudinal cross sectional view of the expanding plug flow reactor of FIG. 2, showing the depth of the medium in the conduit; and

FIG. 4 is a schematic view for an alternate embodiment of a system in accordance with the present invention.

### DESCRIPTION OF THE PREFERRED EMBODIMENTS

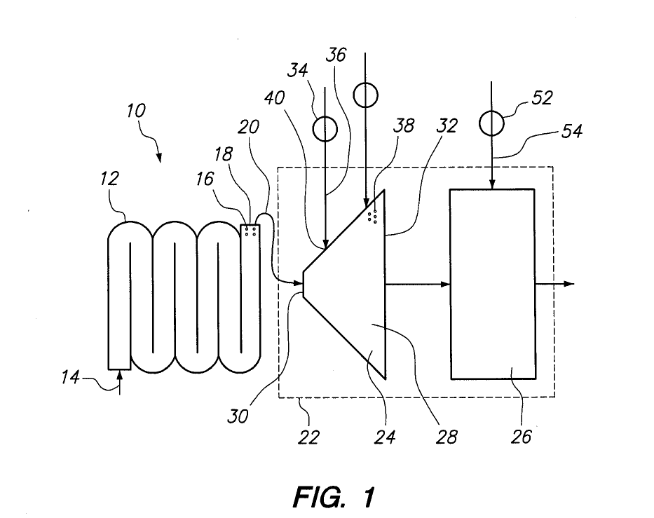

Referring initially to FIG. 1, a system for growing selected algae cells is shown, and is generally designated 10. As shown in FIG. 1, the system 10 includes a closed reactor 12, such as a continuous flow photobioreactor. As shown in FIG. 1, the closed reactor 12 is fed with an inoculum medium 14 and continuously grows an inoculum of algae 16. As the inoculum of algae 16 reaches the end 18 of the closed reactor 12, it is at full concentration. Then, the inoculum of algae 16 passes out of the closed reactor 12 in an effluence (arrow 20).

As shown in FIG. 1, the effluence 20 containing the inoculum of algae 16 passes from the closed reactor 12 to an open system 22, such as an open raceway. In FIG. 1, it can be seen that the open system 22 comprises an expanding plug flow reactor (EPFR) 24 and a standard plug flow reactor (SPFR) 26. Structurally, the EPFR 24 includes a conduit 28 with a first end 30 for receiving the effluence 20 and a second end 32. Further, the open system 22 includes a pump 34. As the effluence 20 enters the EPFR 24, the pump 34 adds a growth medium (arrow 36) to the EPFR 24 to dilute the concentration of algae 38 within the EPFR 24 to about 0.5 grams per liter of fluid. Further, the growth medium 36 includes the nutrients necessary to support the desired growth of the algae 38. As shown in FIG. 1, the open system 22 may include a plurality of pumps 34 for feeding the growth medium 36 at locations 40 along the length of the EPFR 24.

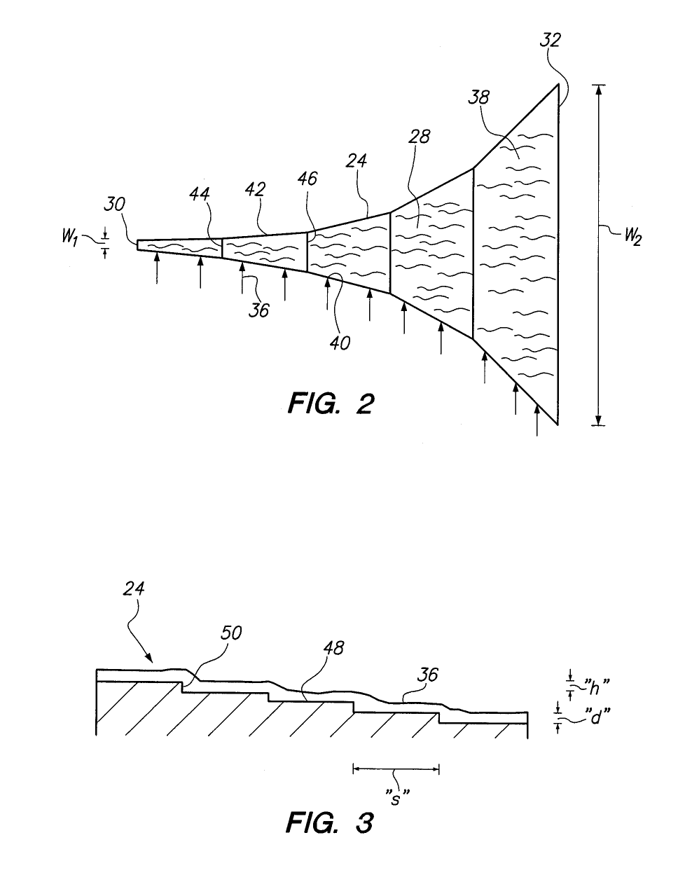

Referring now to FIG. 2, the structure and operation of the EPFR 24 may be understood. As shown, the first end 30 of the EPFR 24 has a width W₁ and the second end 32 of the EPFR 24 has a width W₂ that is substantially greater than W₁. In FIG. 2, the EPFR 24 is not drawn to scale. In certain embodiments, W₁ will equal ten feet, while W₂ will equal 300 feet. Further, the EPFR 24 can be seen to include a plurality of sections 42. Further each section 42 expands in width from its proximal end 44 to its distal end 46. As shown, the width of each section 42 doubles from its proximal end 44 to its distal end 46. As a result, the EPFR 24 has a substantially logarithmic increase in width. While FIG. 2 illustrates an increase in width for each successive section, it is envisioned that sections 42 having a constant width could be interspersed among the widening sections 42.

Importantly, the fluid medium 36 and algae 38 flow through the EPFR 24 under the influence of gravity. For purposes of the present invention, this gravity flow is accomplished using a structured gradient. A preferred embodiment of a structured gradient for use with the EPFR 24 is shown in FIG. 3. There it will be seen that the floor 48 of the conduit 28 is formed with a plurality of steps 50. In detail, the steps 50 are defined by a height “h” of approximately 3 centimeters, with a distance “s” between the steps 50 being preferably on the order of approximately 100 meters. Typically, the EPFR 24 may be over 1000 meters long and the algae 38 may have a residence time of about thirty days in the EPFR 24.

An important aspect of the EPFR 24 for the present invention will be appreciated with reference to FIG. 3. This aspect is that the depth “d” of the fluid medium 36 in the conduit 28 needs to be rather shallow (i.e. less than about 15 cm, and preferably around 7.5 cm). To maintain this depth “d”, however, it is necessary to add the fluid medium 36 along the length of the EPFR 24 as the EPFR 24 widens. Importantly, the increase in width among EPFR sections 42 allows for logarithmic growth of the algae 38 while the concentration of the algae 38 is maintained at the high concentration of at least 0.5 grams per liter.

In cross-reference to FIGS. 1 and 2, as the medium 36 and algae 38 reach the second end 32 of the EPFR 24, they are transferred to the SPFR 26. At this stage, the algae 38 stops growing and, instead, begins to produce oils to store energy. In order to instigate oil production in the algae 38, a pump 52 may introduce a trigger medium 54 into the SPFR 26. Specifically, the trigger medium 54 may lack a desired nutrient, such as nitrogen or phosphorus, which causes the algae 38 to produce oil. Alternatively, the SPFR 26 may receive only the medium 36 and algae 38 from the EPFR 24, without any additional medium 54. In either case, oil production in the algae 38 is triggered by the lack of nutrients to support growth.

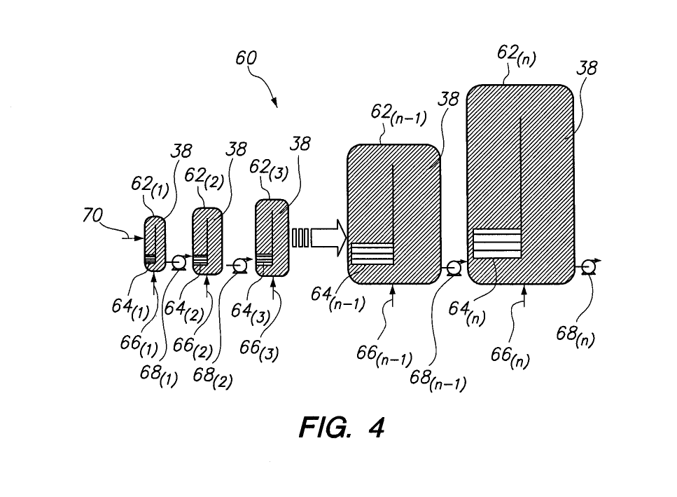

In FIG. 4, an alternate embodiment for the present invention is shown and is generally designated 60. As shown, the system 60 includes an “n” number of open ponds 62 with the smallest open pond 62₍₁₎ being designated as the “first upstream pond”, and the largest open pond 62₍ₙ₎ being designated as the “last downstream pond”. Intermediate open ponds 62 are arranged in order, according to size, with an exponentially increasing surface area in a downstream direction. In this case, the downstream direction extends from the first upstream pond 62₍₁₎ to the last downstream pond 62₍ₙ₎. For the system 60, the ratio between adjacent surface areas of respective open ponds 62 is established by a fixed multiplier. Importantly, this fixed multiplier is determined by the growth rate of the particular algae 38 that are to be cultivated in the system 60.

For the present invention, it is to be appreciated that all of the open ponds 62 in the system 60 are substantially similar to each other. The exception here is only in the size of their respective surface areas. Accordingly, each pond 62 will have a fluid circulating device 64 that is provided for moving (stirring) algae 38 around in the pond 62. Functionally, this is done to promote the growth of algae 38 while there is a culture of the algae 38 in the particular open pond 62. Examples for a suitable fluid circulating device 64 would be a standard circulation pump or a paddle wheel. Both of these types of devices are well known in the pertinent art.

It will also be seen in FIG. 4 that each open pond 62 has a medium addition conduit (represented by arrow 66) which is provided to add medium into the respective open pond 62, as needed. Further, the open ponds 62 are connected via respective transfer conduits for selective communication with each other. For example, the upstream open pond 62₍ₙ₋₁₎ is connected in fluid communication via a transfer conduit with its adjacent downstream open pond 62₍ₙ₎. Preferably, the transfer conduits are transfer pumps 68. As shown in FIG. 4, the transfer conduit between open pond 62₍ₙ₋₁₎ and open pond 62₍ₙ₎ is a transfer pump 68₍ₙ₋₁₎. As implied above, however, this particular structure is only exemplary. As an alternative to using transfer pumps 68, the open ponds 62 in system 60 can be terraced to provide for a gravity flow of liquid between the various pairs of upstream and downstream open ponds 62.

In addition to the specific structural components of the system 60 described above, inoculum algae 16 in an inoculum medium 14 can be fed into the first upstream open pond 62₍₁₎ via a first transfer conduit (represented by the arrow 70). At the downstream end of the system 60, after traversing the system 60, the now fully grown algae 38 can be removed from the last downstream open pond 62₍ₙ₎ via a last transfer conduit (e.g. transfer pump 68₍ₙ₎.

In the operation of the system 60, algae 38 are progressively grown as they are selectively passed from one open pond 62 to another. The actual time spent by the algae 38 in each open pond 62 in the series will be substantially the same, and will depend on the type of algae 38 that is being cultivated. As a practical matter, the time spent by algae 38 in a particular open pond 62 can be as much as several (e.g. 3) days. In the event, the transfer of algae 38 through the system 60 is done methodically. And preferably, the transfer will be accomplished at nighttime when the growth of algae 38 is delayed due to a lack of sun light.

A transfer sequence for moving algae 38 through the system 60 begins by first emptying the last downstream pond 62₍ₙ₎. To do this, the fully grown algae 38 therein are transferred through a transfer conduit (e.g. transfer pump 68₍ₙ₎) to an oil formation pond (i.e. SPFR 26). Next, the contents of the adjacent upstream open pond 62₍ₙ₋₁₎ are then emptied into the now-empty last downstream open pond 62₍ₙ₎. At this time, additional medium can be added to the last downstream open pond 62₍ₙ₎ via the medium addition conduit 66₍ₙ₎. Specifically, this is done to establish proper conditions for further growth of algae 38 in the open pond 62₍ₙ₎. In turn, the contents of open pond 62₍ₙ₋₂₎ (not shown) are emptied into open pond 62₍ₙ₋₁₎, and an appropriate amount of medium is added. This continues, in sequence, with the contents of each upstream open pond (e.g. pond 62₍₂₎) being transferred into the just-emptied adjacent downstream open pond (e.g. pond 62₍₃₎). The transfer sequence finally ends when the contents of the first upstream open pond 62₍₁₎ have been emptied into open pond 62₍₂₎ and the now-empty upstream open pond 62₍₁₎ has been refilled with inoculum of algae 16. The system 60 then continues to grow algae 38 in respective open ponds 62 until another transfer sequence is initiated.

While the particular Method and System for Growing Microalgae in an Expanding Plug Flow Reactor as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for growing algae cells comprising the steps of:
providing an open system comprising an expanding plug flow reactor (EPFR) for facilitating logarithmic growth of an inoculum of algae cells, and a standard plug flow reactor (SPFR) for treating the algae cells to activate oil production therein;
introducing an inoculum of algae cells into a first end of the EPFR, wherein the EPFR has a second end, and wherein the first end has a width W₁ and the second end has a width W₂, with W₂>W₁;
adding a growth medium at a plurality of locations dispersed between the first end and the second end of the EPFR to support logarithmic growth of the algae cells therein, wherein a selected depth of medium is maintained in the EPFR;
transferring the algae cells from the second end of the EPFR to the SPFR; and
triggering the algae cells in the SPFR to activate oil production.

2. A method as recited in claim 1 wherein the concentration of algae cells in the medium in the EPFR is maintained at approximately 0.5 grams per 1 liter.

3. A method as recited in claim 1 wherein the depth of the medium in the EPFR is less than approximately fifteen inches.

4. A method as recited in claim 1 wherein the EPFR has a structured downstream gradient to move the growth medium and algae cells from the first end to the second end.

5. A method as recited in claim 1 wherein the algae cells have residence time of about thirty days in the EPFR.

6. A method as recited in claim 1 further comprising the steps of:
determining the amount of nutrients required to support growth of the algae cells from a first location to a second location in the EPFR, wherein the width of the EPFR at the second location is greater than the width of the EPFR at the first location;
periodically determining a growth rate for the algae cells at the first location in the EPFR;
ascertaining the duration of time needed for the algae cells at the first location in the EPFR to grow in view of the determined growth rate;
calculating a volumetric flow rate appropriate for moving the algae cells at the first location to the second location after the needed duration of time; and
adding the growth medium between the first location and the second location to cause the algae cells to move at the calculated volumetric flow rate, with the growth medium containing the determined amount of nutrients to support growth of the algae cells from the first location to the second location.

7. A method for growing selected algae cells in an open system comprising the steps of:
providing a system comprising a closed reactor for growing an inoculum of algae cells, an expanding plug flow reactor (EPFR) for facilitating logarithmic growth of the inoculum of algae cells, and a standard plug flow reactor (SPFR) for treating the algae cells to activate oil production therein;
feeding an inoculation medium with a nutrient mix for facilitating growth of the inoculum of algae cells;
passing an effluence containing the inoculum of algae cells from the closed reactor to a first end of the EPFR, wherein the EPFR has a second end, and wherein the first end has a width W₁ and the second end has a width W₂, with W₂>W₁;
adding a growth medium at a plurality of locations dispersed between the first end and the second end of the EPFR to support logarithmic growth of the algae cells therein, wherein a selected depth of medium is maintained in the EPFR;
transferring the algae cells from the second end of the EPFR to the SPFR; and
supplying the SPFR with a trigger medium to activate oil production in the algae cells therein.

8. A method as recited in claim 7 wherein the closed reactor is a continuous flow reactor.

9. A method as recited in claim 7 wherein the concentration of algae cells in the medium in the EPFR is diluted and maintained at approximately 0.5 grams per 1 liter.

10. A method as recited in claim 7 wherein the depth of the medium in the EPFR is less than approximately fifteen inches.

11. A method as recited in claim 7 wherein the EPFR has a structured downstream gradient to move the growth medium and algae cells from the first end to the second end.

12. A method as recited in claim 7 wherein the algae cells have residence time of about thirty days in the EPFR.

13. A method as recited in claim 7 further comprising the steps of:
determining the amount of nutrients required to support growth of the algae cells from a first location to a second location in the EPFR, wherein the width of the EPFR at the second location is greater than the width of the EPFR at the first location;
periodically determining a growth rate for the algae cells at the first location in the EPFR;
ascertaining the duration of time needed for the algae cells at the first location in the EPFR to grow in view of the determined growth rate;
calculating a volumetric flow rate appropriate for moving the algae cells at the first location to the second location after the needed duration of time; and
adding the growth medium between the first location and the second location to cause the algae cells to move at the calculated volumetric flow rate, with the growth medium containing the determined amount of nutrients to support growth of the algae cells from the first location to the second location.

14. A system for growing algae cells which comprises:
a source of an inoculum of algae cells;
a plurality of open ponds connected in selective fluid communication with each other, with the plurality of open ponds arranged in sequence from a first upstream pond to a last downstream pond, wherein each downstream pond has an exponentially greater surface area relative to its adjacent upstream pond;
a first transfer conduit for transferring the inoculum from the source to the first upstream pond to create a culture therein for algae growth and to provide for a subsequent transfer of the culture therefrom to successive downstream ponds for further algae growth; and
a last transfer conduit for selectively transferring fully grown algae cells from the last downstream pond to an oil formation pond.

15. A system as recited in claim 14 wherein each open pond comprises:
a fluid circulating device to establish liquid flow in the pond;
a medium addition conduit for adding medium into the culture in the pond; and
a transfer pump for transferring the culture downstream from the pond.

16. A system as recited in claim 14 wherein a fixed multiplier is determined to establish a ratio of a surface area for each pond relative to a surface area of an adjacent pond, and wherein the multiplier is determined by a growth rate of the algae used for cultivation.

17. A system as recited in claim 14 wherein the depth of liquid in each pond is less than fifteen inches, and the residence time of the culture in each pond is less than three days.

18. A system as recited in claim 14 wherein a transfer of culture from a specified pond is completed, and the specified pond is substantially emptied, before the empty pond receives a transfer of culture from an adjacent upstream pond.

19. A system as recited in claim 18 wherein the transfer of culture is accomplished during the nighttime.

20. A system for growing algae cells comprising:
a closed reactor for growing an inoculum of algae cells;
an open system comprising an expanding plug flow reactor (EPFR) for facilitating logarithmic growth of the inoculum of algae cells, and a standard plug flow reactor (SPFR) for treating the algae cells to activate oil production therein;
a means for passing an effluence containing the inoculum of algae cells from the closed reactor to a first end of the EPFR, wherein the EPFR has a second end, wherein the first end has a width W₁ and the second end has a width W₂, and wherein W₂>W₁;
a means for adding a growth medium at a plurality of locations dispersed between the first end and the second end of the EPFR to support logarithmic growth of the algae cells therein, wherein the adding means maintains a selected depth of medium in the EPFR; and
a means for transferring the algae cells from the second end of the EPFR to the SPFR.
